(19) United States
(12) Patent Application Publication
(10) Pub. No.: US 2022/0110837 A1
(43) Pub. Date: Apr. 14, 2022

(54) THERAPEUTIC COMPOSITION FOR ARRESTING, PREVENTING AND REVERSING DENTAL DISEASE

(71) Applicant: MOHAMED HARUNANI, ROCKFORD, IL (US)

(72) Inventor: MOHAMED HARUNANI, ROCKFORD, IL (US)

(21) Appl. No.: 17/190,532

(22) Filed: Mar. 3, 2021

Related U.S. Application Data
(60) Provisional application No. 63/091,310, filed on Oct. 14, 2020.

(51) Int. Cl. A61K 8/02; A61K 8/21; A61K 8/19; A61Q 11/00; A61K 8/49; A61K 8/24; A61K 8/36; A61K 8/9794; A61K 8/34; A61K 8/99; A61K 33/16; A61K 45/06; A61K 33/06; A61K 33/10; A61K 47/02; A61K 47/10; A61K 47/46; A61K 9/00; A61K 31/52; A23G 3/36; A23G 3/42; A23G 3/56; A23L 33/16; A23L 33/135; A23L 33/125; A23L 33/15; A23L 33/22; A23L 33/00

(52) U.S. Cl. CPC A61K 8/0216; A23V 2002/00; A61K 8/19; A61Q 11/00; A61K 8/4953; A61K 8/24; A61K 8/36; A61K 8/9794; A61K 8/345; A61K 8/99; A61K 33/16; A61K 45/06; A61K 33/06; A61K 33/10; A61K 47/02; A61K 47/10; A61K 47/46; A61K 9/0056; A61K 31/52; A23G 3/362; A23G 3/368; A23G 3/366; A23G 3/42; A23G 3/563; A23L 33/16; A23L 33/135; A23L 33/125; A23L 33/15; A23L 33/22; A23L 33/40; A61K 8/21

(57) ABSTRACT

The present invention discloses a non-acidic-candy based composition for arresting or reversing dental caries by transforming the oral cavity from an acidic, bacteria-filled environment to an environment more conducive to remineralization by quickly neutralizing the acids recreated by ingestion of acidic compounds or by cariogenic bacteria while supplying necessary ions for tooth remineralization. This improves oral health, prevents pain and suffering, and serves as a conduit to deliver additional therapeutic agents. The composition comprises any combination of a calcium-based compound, a bicarbonate compound, a fluoride compound, an active ingredient, a therapeutic agent based on need, a non-cariogenic sweetener, a hydrating agent, at least one additive, and water. The composition is a candy that has a non-acidic-based composition which neutralizes acids while delivering necessary ions in a pH optimized environment to create the best possible conditions needed to arrest and/or reverse disease processes in the oral cavity and beyond.

### FIELD OF THE DISCLOSURE

The present invention generally relates to a therapeutic composition for arresting, preventing and reversing dental disease. More specifically, the present invention relates to a non-acidic-based composition for improving oral health and delivering nutrients, agents, or combinations thereof.

### BACKGROUND OF THE DISCLOSURE

Oral hygiene is one of the most important aspects of personal care among consumers. Consumers worldwide use different types of products for oral care as a part of maintaining dental hygiene. Not all oral disease is from poor oral hygiene—in fact, in the adult, lack of saliva due to medications, head, and neck radiation treatment, and Sjogren's syndrome are significant causes. Poor oral hygiene and increased sugar consumption keep the oral cavity's pH low (acidic) and produce demineralization or tooth decay. Decay, gum disease, and discolored teeth are all symptoms of poor oral health. Generally, the teeth are composed of a hard enamel outer layer and a softer inner dentin layer which covers the pulp (composed of a complex network of nerves, blood vessels, and lymphatics). The layers are arranged in a crystalline structure called hydroxyapatite composed of calcium and phosphate ions and contain trace minerals such as strontium, magnesium, lead, and fluoride. The enamel is formed in rods within a predominantly inorganic matrix material containing hydroxyapatite, protein, and other compounds. Dentin has an increased type I collagen content and decreased mineral content when compared to enamel. Dentin contains fluid-filled channels (tubules) and decays more rapidly than enamel.

A succession of oral bacteria produces decay by forming plaque deposits on the teeth. For example, the bacterium Streptococcus produces glucan that helps bacteria adhere to teeth and form plaque deposits. Initial colonization by S. mutans is followed by a succession of bacteria that produce increasing amounts of lactic acid causing the removal of minerals (demineralization) from the tooth and consequently weakening it. Plaque bacteria metabolize sugars (fructose, sucrose, and glucose) and carbohydrates, resulting in the formation and secretion of lactic acid as a byproduct of their metabolization. At 5.5 pH, demineralization (also called cavitation or decay) begins and continues through the enamel until dentin is penetrated. Since dentin dissolves at a higher pH of 6.2-6.5 (less acidic environment), the decay process speeds up as decay proceeds. Tooth enamel is soluble in acids. During the demineralization process, minerals (calcium ions and phosphate ions) are removed from the enamel and dentin until the enamel collapses. In addition, the defect created cannot be replaced by remineralization. Cycles of remineralization and demineralization continue throughout the decay process with demineralization created by the ingestion of acidic food/drinks and by acid-forming bacteria resulting in an acidic oral environment while remineralization (replacement of the removed mineral from saliva) occurring during periods of neutral pH. Enamel may be remineralized between meals by replacing missing calcium and phosphate ions during a neutral pH (7 or higher) by ions present in the saliva. The presence of fluoride hastens this process. Saliva is necessary for repairing tooth structure and inhibiting tooth decay. Saliva production is essential to several biological functions including swallowing, digestion, and lubrication of the oral cavity. Salivary glands may become impaired and/or damaged due to accident, trauma, illness, disease, and/or medical treatments, including surgery, medications, radiation therapy, and chemotherapy treatments. Decreased saliva is the largest cause of oral issues in the elderly; it causes tooth decay, tooth loss, dental abscesses, and mucositis. Other significant health conditions, including malnutrition, pain, and/or infections, can also be related to a decreased salivary flow and/or altered oral environment.

Further, a common scenario and cause of increased tooth decay involves the usage of bottled water by children and adults, which most often is not fluoridated. This results in two consequences, which include the unavailability of fluoride in the teeth of the growing children, and the lack of fluoride does not allow for the strengthening of the surface layer, and so these teeth are more prone to breakdown. Fluoride converts as the hydroxyapatite crystal to fluoro hydroxyapatite crystal, which is much less soluble. Fluorapatite (FA) is formed when fluoride exchanges with the hydroxyl groups of present hydroxyapatite crystals in the tooth, which is less soluble in acid.

Further, in nursing homes and homes for the disabled, the most common foods are soft and sticky to the teeth. As known, any carbohydrates and/or sugars are converted to lactic acid by the cariogenic bacteria. Since these teeth do not get brushed immediately, they have a tendency to decay rapidly. In these populations, manual dexterity is also a problem, making brushing more difficult. Further, average life of restorations (fillings) is less than ten years. In young patients, these restorations may need to be replaced multiple times at a considerable cost in terms of money, time, pain and suffering.

In a study of 380 beverages purchased from stores in Birmingham, Ala., categorized (e.g. juices, sodas) and assessed for pH, It was found that the majority (93%, 355/380) of beverages had a pH below 4.0 and 7% (25/380) had a pH≥4. Relative beverage erosivity zones based on previous studies of apatite solubility in acid indicated: 39% (150/380) of the beverages were considered extremely erosive (pH<3.0); 54% (205/380) were considered erosive (pH 3.0 to 3.99); and 7% (25/380) were considered minimally erosive (pH 4.0). These drinks usually contain citric acid, which is one of the worst of edible acids due to its pKa.

Dental erosion from beverages is primarily caused by either phosphoric acid and/or citric acid, and both are triprotic acids with 3 available [H+] enabling proton-promoted dissolution. Chelation or ligand-promoted dissolution by anionic citrate contributes to enamel demineralization by the removal of Ca++ at a higher pH range approaching 6. At the erosive pH, only 3% of citrate ions are appropriately ionized to chelate Ca++, indicating their contribution to the erosive process at this pH is minimal. However, if anionic citrate remains within the oral cavity for extended time intervals allowing the pH to rise to 6, chelation could play a contributing role in the erosive process; such as the eating of citrus fruits more than twice a day has been associated with dental erosion. Nevertheless, high concentrations of [H+] reflected by low pH from citric and/or phosphoric acid result in undersaturation for fluor- and hydroxyapatite leading to dental erosion. Hence, pH is the controlling parameter in determining the erosive potential of beverages.

Further, the present society consumes numerous meals on the run and fast food or in their vehicle with no way of cleaning or de-acidifying their mouth to stop the acid demineralization, whether it is from the acidic food/drink or the lactic acid being produced by the cariogenic bacteria. Additionally, most medications cause dry mouth and a reduction in the salivary flow, which lets the acid stay in the mouth longer and having a longer time to dimineralize.

Several attempts have been made to address the issues related to improving oral health. One such example is disclosed in a United States granted patent No. 8518383 entitled “Oral care products comprising buffer systems for improved mineralization/remineralization benefits” (the “'383 Publication”). The “'383 Publication discloses a product for improved mineralization/remineralization of teeth. The oral product is comprised of calcium, phosphate, sodium, and potassium salts. This oral product does not address the acid issue, nor contains fluoride, which is the catalyst needed for remineralization.

Another example is disclosed in a United States granted patent No: 4906455 entitled “Method for treating xerostomia employing chewing gum containing relatively insoluble, hydrophobic, food-grade organic acid” (the “'455 Publication”). The “'455 Publication discloses a method for treating xerostomia, dry mouth, and employing a specially formulated chewing gum. The method involves chewing a sugar-sugarless gum that contains a relatively insoluble, hydrophobic, food-grade organic acid, such as adipic acid. Chewing gum gradually releases the acid at a desirable linear rate for about 20 to 30 minutes. Since acids produce a sour taste that stimulates any remaining salivary gland to increase saliva output, the slow release of the acid from the gum results in an increase in the salivary flow rate in xerostomia patients having decreased salivary gland function. Unfortunately, this acid release is not constant, and it also lowers pH resulting in increases demineralization.

Yet another example is disclosed in a United States granted patent No: 5698215 entitled “Chewing gum composition with fluoride and citric acid” (the “'215 Publication”). The “'215 Publication discloses a chewing gum which possesses fluoride and citric acid for improved dental health. The citric acid is incorporated to provide a means by which fluoride can be absorbed into the tooth structure. The mechanism of this product is the following: citric acid microscopically opens pores in the enamel; this opening of the pores, at the enamel surface, enables greater contact and penetration of the fluoride to be absorbed into the enamel. The resultant effect is greater penetration of fluoride into the enamel and the underlying tooth structure (dentin). Citric acid has one of the highest pKa and is one of the most destructive to the teeth of all the edible acids. Unfortunately, since citric acid lowers pH, it also increases demineralization. This can also etch the surface of teeth and dental restorations, thereby ruining their integrity.

Yet another example is disclosed in a United States granted patent No: 5702687 entitled “Chewing gum product with plaque-inhibiting benefits” (the “'687 Publication”). The '687 Publication discloses a chewing gum product containing two or more active ingredients for improved dental health. The chewing gum product contains ingredients that include a gum base, dispersed particles of organic-encapsulated sodium bicarbonate, a bulking sweetener, a plaque-inhibiting ingredient, and a flavorant. The claimed benefit is from the higher pH created by the chewing gum. The bicarbonate release cannot be constant or prolonged in chewing gum, and increasing the pH alone does not cause remineralization.

Yet another example is disclosed in a United States granted patent No: 5993786 entitled “Anti-carious chewing gums, candies, gels, toothpastes and dentifrices” (the “'786 Publication”). The '786 Publication discloses chewing gums, candies, confectioneries, toothpaste, dentifrices, and gels containing non-toxic sparingly soluble calcium and phosphate compounds as additives, causing the release of calcium and phosphate ions into the oral cavity gradually and persistently for a period no less than 5 minutes. The '786 Publication provides released calcium phosphate ions that diffuse into partially demineralized tooth enamel or dentin, leading to remineralization and repair of caries lesions, reducing dental plaque, and plugging open dentinal tubules, which decreases tooth cold sensitivity from exposed dentin. Thus, it is claimed that the invention provides agents and methods for remineralization of teeth and for reducing or eradicating cariogenic challenge in plaque following sucrose intake. The formulations of the invention can thereby produce effective anticaries actions without fluoride. In addition, the formulations of this invention can be used to desensitize hypersensitive teeth. Unfortunately, the contact time is too short to create a neutral pH and to incorporate the calcium and phosphate into the tooth. Additionally, recent studies have shown that including fluoride significantly speeds up this process.

Although the above-discussed disclosures may help to improve oral health, they have limitations. For instance, they do not reduce acidity in the oral cavity at a rate conducive to decreasing and maintaining the acidity for a duration long enough for remineralization a necessity to stop demineralization and promote remineralization utilizing hard and/or soft candy. Furthermore, most other materials do not neutralize the pH and release all the ions that are essential for promoting remineralization. Remineralization occurs during periods of neutral pH, requiring a buffer; this process is further complicated by the short duration of chewing gum or dentifrice in the oral cavity.

Accordingly, there is a need for a composition that could improve oral health by neutralizing the oral pH, repairing damaged tooth structure, strengthening the enamel, and delivering nutrients, therapeutic agents, or combinations to improve health and well-being. Further, there is a need for a composition in a form candy that would immediately reduce acidity, encourages salivary flow and natural sucking and other movements that occur, and causes the cleansing of the mouth and thus enable reduction of the pathogenic load while encouraging remineralization. Further, there is a need for a low cost, composition in the form of candy that would naturally and permanently reverses incipient lesions without the need for a dental restoration. Further, there is a need for a composition that would quickly reduce acidity and encourages remineralization.

### BRIEF SUMMARY OF THE DISCLOSURE

The first objective of the present invention is to provide a non-acidic-based buffering composition for improving oral and general health by delivering nutrients, therapeutic agents, or combinations thereof.

An objective of the present invention to provide a nonacidic-based composition that could create an oral environment that arrests and/or reverses disease processes such as gum disease and tooth decay.

It is another objective of the present invention to provide a composition configured to neutralize pH in the oral cavity quickly and simultaneously to deliver ions that are necessary to create an oral environment that reverses disease processes in the oral cavity.

It is yet another objective of the present invention to provide a composition configured to decrease demineralization which weakens teeth.

It is yet another objective of the present invention to provide a composition that provides ions necessary for tooth remineralization.

It is yet another objective of the present invention to provide a composition that provides fluoride to help with remineralization and inhibit or reduce bacterial growth.

It is yet another objective of the present invention to provide a composition in candy with a zero glycemic index that allows it to be consumed safely by children, adolescents, adults, and diabetics.

In one formulation of the candy, the composition contains at least one calcium releasing compound, at least one bicarbonate compound, at least one active ingredient, at least one non-cariogenic sweetener (like erythritol), at least one high-intensity sweetener, at least one hydrating agent, and at least one additive. The composition further comprises water, a colorant, and a flavorant. In one embodiment, the additive may be an antimicrobial agent, an antibacterial agent, an antiviral agent, a tooth whitening agent, a probiotic, an energy supplement, or caffeine. In another embodiment, the composition further comprises at least one of vitamin, mineral, or electrolyte.

In another embodiment, the composition comprises at least one high-intensity sweetener component in a range of about 0.001 g to about 0.006 g. In yet another embodiment, the composition comprises fiber like nutriose of about 2-6 g, and β-tricalcium phosphate (β-TCP) in a range of about 0.5 g to about 5 g, and approximately 1-6 g of a sweetener like an erythritol. In yet another composition, the mixture may include a fluoride-based compound ranging from 0 g to about 0.2 g, about 0-5 water, bicarbonate ranging from about 0.02 to about 20 mg, and probiotic compound in a range will vary. In one embodiment, the composition can be made in the form of a hard candy, a chewy candy, a gummy candy, a jelly candy, or a lollypop type candy.

Features and advantages of the subject matter hereof will become more apparent in light of the following detailed description of selected composition types, as illustrated in the accompanying FIGURES. To demonstrate examples of the components of the candy delivery system and its possible modifications, figures, and tables are used to illustrate different types of uses and composition.

### BRIEF DESCRIPTION OF THE DRAWINGS

The present subject matter will now be described in detail with reference to the drawings, provided as illustrative examples of the candy to enable those skilled in the art to practice the subject matter. It will be noted that throughout the appended drawings or tables the FIGURES and examples are not meant to limit the scope of the candy to a specific present subject matter or to a single composition but it is designed to be composed of the same ingredients with different concentrations. By using the candy delivery system and varying the composition of various brands of candy are possible by way of interchange of some or all of the described or illustrated elements and, further, wherein:

FIG. 1 exemplarily illustrates a non-acidic-based composition and the composition is in the form of hard or soft candy or lollypop (100), according to an embodiment of the present invention;

FIG. 2 exemplarily illustrates an end-user (102) having improved oral health on using the non-acidic-based composition, according to an embodiment of the present invention; and

TABLE 1 exemplarily illustrates a non-acidic-based composition is in the form of hard or soft candy or lollypop (100).

### DETAILED DESCRIPTION OF THE EMBODIMENTS

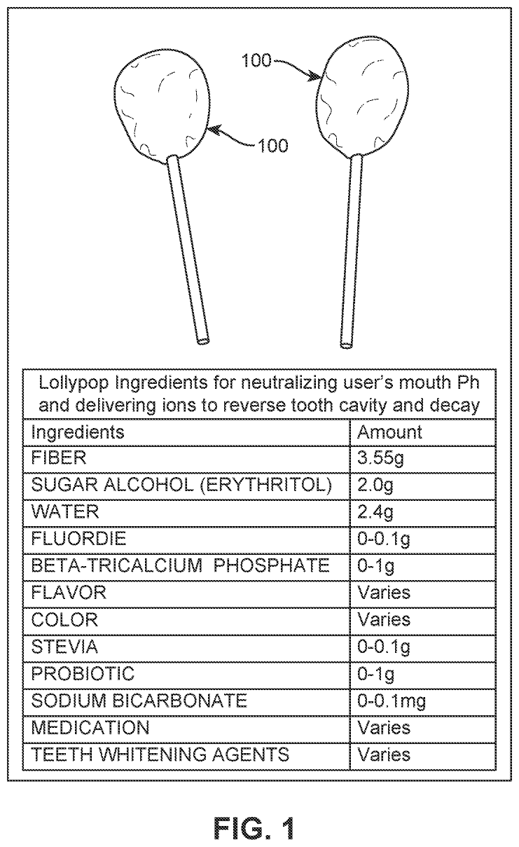

The detailed description seen in FIG. 1 is intended as a description of exemplary embodiments in which the presently disclosed subject matter may be practiced. The term “exemplary” used throughout this description means “serving as an example, instance, or illustration,” and should not necessarily be construed as preferred or advantageous over other embodiments. The detailed description includes specific details for providing a thorough understanding of the presently disclosed composition. However, it will be apparent to those skilled in the art that the presently disclosed subject matter may be practiced without these specific details.

In the present specification, an embodiment showing a singular component should not be considered limiting. Rather, the subject matter preferably encompasses other embodiments including a plurality of the same component, and vice-versa, unless explicitly stated otherwise herein. Moreover, the applicant does not intend for any term in the specification or claims to be ascribed an uncommon or special meaning unless explicitly set forth as such. Further, the present subject matter encompasses present and future known equivalents to the known components referred to herein by way of illustration. The composition will vary according to the desired therapeutic outcome.

Although the present disclosure describes a non-acidic-based composition for improving oral health and delivering nutrients, therapeutic agents, or combinations thereof, it is to be further understood that numerous changes may arise in the details of the embodiments of the composition. It is contemplated that all such changes and additional embodiments are within the spirit and true scope of this disclosure.

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure.

The present invention discloses a non-acidic-based composition for improving oral health and delivering nutrients, therapeutic agents, or combinations thereof. The composition, immediately upon use, lowers the acidity in the oral cavity to create an environment that arrests or reverses disease processes (i.e., dental caries and periodontal disease) while bringing other benefits to the consumer including, but not limited to, stronger bones and whiter, healthier teeth. The composition is in the form of a non-sugar (sucrose, fructose, or glucose) containing candy with the capacity to deliver various ingredients in a pH-controlled manner, to quickly decrease the acidity of the mouth and deliver the ingredients needed to prevent and/or repair teeth and also provide other therapeutic benefits in a beneficial and/or less destructive manner.

In one embodiment, the composition comprises at least one calcium-based compound, at least one bicarbonate compound, at least one active ingredient, at least one sweetener, at least one hydrating agent, and at least one additive. The composition further comprises water. In one embodiment, the additive is selected from the group consisting of an antimicrobial agent, an antibacterial agent, an antiviral agent, a tooth whitening agent, a probiotic, an energy supplement, a colorant, caffeine, and a flavorant. In another mixture of encapsulated components, the composition includes at least one of vitamin, mineral, or electrolyte. In one embodiment, the additive is an agent that helps with halitosis/mal-odor (a lactobacillus or other).

Referring to TABLE 1, the composition includes at least one high-intensity sweetener component in a range of about 0.001 g to about 0.006 g. In yet another embodiment, the composition comprises fiber of about 2-6 g, and β-tricalcium phosphate (β-TCP) in a range of about 0.5 g to about 5 g, and approximately 1-6 g of a sweetener like an erythritol. In yet another composition, the mixture may include a fluoride-based compound ranging from 0 g to about 0.2 g, about 0-5 water, bicarbonate ranging from about 0.02 to about 20 mg, and the amount of probiotic and other therapeu-therapeutic compounds will vary. In one embodiment, the composition is in a form of lollypop (100). In other embodiments, the candy composition can be made in a form of a hard candy, a chewy candy, a gummy candy, or a jelly candy.

In one embodiment, the candy is a lollypop, and its composition comprises fiber like nutriose of about 4.46 g, sugar alcohol of about 2.52 g, water of about 1.05 g, a fluoride-based compound of about 0.725 g, β-tri-Calcium phosphate of about 0.75 g, high-intensity sweetener compound like Stevia of about 0.038 g, sodium bicarbonate ranging from about 1 mg, a flavorant of about 0.45 g and a colorant.

Active ingredients in each embodiment will vary based on therapeutic needs. For example, the fluoride-based compound forms harder enamel and inhibits decay; the tooth whitening agents contain an ingredient which will whiten teeth; the probiotics inhibit bacteria causing gum disease; the antiviral and/or antibacterial agents reduce the pathogenicity of the oral cavity, and the vitamins and minerals (the building blocks of the tooth) strengthen teeth and improve oral and general health.

Today, electrolytes are generally consumed from sports drinks, which are very acidic. This is a great way to intake electrolytes without destructive sugar and/or acid. Similarly, caffeine and other energy-boosting ingredients can be provided without the sugars and acids that increase tooth decay. The addition of bicarbonate can further help buffer against the acids created by consuming acids and/or plaque containing bacteria. In addition, the candy composic-omposition aids therapeutic agent delivery.

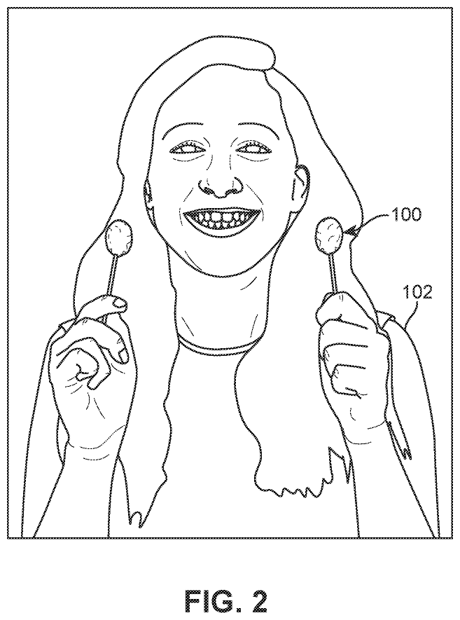

FIG. 2 exemplarily illustrates an end-user (102) with improved oral health using the non-acidic-based composition, according to an embodiment of the present invention. The candy dissolves in the oral cavity quickly decreases oral acidity and releases beneficial ingredients. Thereby, the composition creates a favorable oral environment for oral health including remineralization of the tooth, leading to the reversal of cavities and other diseases while also being able to deliver other beneficial ingredients.

In one formulation, the dosage of the therapeutic agents/ingredients of the composition may vary based on desired outcomes, timed release of therapeutic agents, and tailored for subjects of different ages. In different formulations, the candy may be supplied in different flavors and consistencies to appeal to different tastes and unique needs. In addition, the composition is safe for children and diabetics without gastric distress created by alcohol sugars and/or side effects of other sweeteners. Furthermore, it is safe for pets (unlike xylitol containing sweeteners).

In one embodiment, the composition of the present invention may vary depending on the condition being addressed. In another embodiment, the ingredients of the composition are microencapsulated and/or directly mixed into the candy. As the candy is consumed, the therapeutic agent is delivered in a controlled fashion without the harmful effects of sug-sugar and acid commonly associated with candy.

The composition is configured to alter the oral environment by adding elements including, but not limited to, calcium, phosphate, and buffering ions. The composition is configured to deliver ions required for oral health simultaneously while being separated during delivery utilizing microencapsulation. The composition is configured to decrease acidity, increase salivary flow, and offer a tasty and effective means to significantly change the oral environment to quickly stop the demineralization and enhance remineralization.

The composition is configured to quickly reduce the acidity of the oral environment while delivering agents that promote oral and general health. The non-acidic-based composition acts as a vehicle to deliver different therapeutic agent using a candy delivery system that would quickly decrease the acidity of the oral environment, increase saliva flow and improve oral and general health without the adverse effects associated with sugar containing candy in the oral environment.

The non-acidic-based composition is configured to reduce the acidity of the oral environment quickly. In addition, the composition is configured to provide a sustained release of therapeutic calcium, phosphate, and fluoride ions into the oral cavity for the highest therapeutic impact to reduce or eradicate cariogenic challenge in plaque following sucrose intake. In addition, the composition comprises erythritol and other similar sweeteners that have proven to reduce bacteria and plaque in the oral cavity. The released calcium, phosphate, and fluoride ions can diffuse into partially demineralized tooth enamel or dentin, leading to remineralization and repair of the caries lesion. These ions can also precipitate in dentinal tubules and relieve sensitive teeth. The non-acidic-based composition as a delivery system aids compliance and pleasure while improving health and reducing costs to the user, the payor, and the government by reducing the cost of treatment, thus creating significant savings for all in terms of health and wealth while improving wellbeing. This composition is ideal for use after a meal when one cannot brush their teeth, for snacking, and the perfect solution to reduce decay in elderly populations, particularly in a nursing home environment. The life expectancy of dental restoration is about ten years, but a tooth that has been remineralized can last a lifetime without the need for continued treatment.

In the above description, numerous specific details are set forth such as examples of some embodiments, specific components, devices, methods, in order to provide a thorough understanding of embodiments of the present disclosure. It will be apparent to a person of ordinary skill in the art that these specific details need not be employed and should not be construed to limit the scope of the disclosure.

In the development of any actual implementation, numerous implementation-specific decisions must be made to achieve the developer's specific goals, such as compliance with system-related and business-related constraints. Such a de-development effort might be complex and time-consuming, but is nevertheless a routine undertaking of design, fabrication, and manufacture for those of ordinary skill. Hence as various changes could be made in the above constructions without departing from the scope of the invention, it is intended that all matter contained in the above description or shown in the explanation, table, and accompanying drawings shall be interpreted as illustrative and not in a limiting sense.

The foregoing description of embodiments is provided to enable any person skilled in the art to make and use the subject matter. Various modifications to these embodiments will be readily apparent to those skilled in the art, and the novel principles and subject matter disclosed herein may be applied to other embodiments without the use of the innovative faculty. The claimed subject matter set forth in the claims is not intended to be limited to the embodiments shown herein but is to be accorded the widest scope consistent with the principles and novel features disclosed herein. It is contemplated that additional embodiments are within the spirit and true scope of the disclosed subject matter.

What is claimed is:

1. A therapeutic composition for improving oral health and delivering nutrients, other agents, or combinations thereof, comprising:
at least one fluoride-based compound;
at least one calcium-based compound;
at least one acid neutralizing agent;
at least one active ingredient;
at least one tooth whitening agent;
at least one probiotic;
at least one energy supplement;
at least one sweetener;
at least one hydrating agent;
at least one additive, and
at least one agent to assist with halitosis,
wherein said composition is configured to neutralize pH in an oral cavity and delivers ions to create an environment that arrests or reverses disease processes.

2. The composition of claim 1, wherein said additive is selected from a group comprising an antimicrobial agent, an antibacterial agent, an antiviral agent, a colorant, and a flavorant.

3. The composition of claim 1, further comprises at least one vitamin or mineral.

4. The composition of claim 1, further comprises at least one electrolyte.

5. The composition of claim 1, wherein said at least one agent to assist with halitosis, is a bacteria.

6. The composition of claim 1, wherein said neutralizing agent is a bicarbonate.

7. The composition of claim 1, further comprises a stimulant, wherein said stimulant is caffeine.

8. The composition of claim 1, provides the best environment for remineralization, wherein the candy composition comprises of a high-intensity sweetener in a range of about 0.001 g to about 0.006 g, fiber in the range of about 2-6 g, β-tricalcium phosphate (β-TCP) in a range of about 0.5 g-5 g, alcohol-based sweetener in the range of about 1-6 g, fluoride-based compound ranging from 0 g to about 0.2 g, bicarbonate in the range of about 0.02 mg to about 20 mg and water.

9. The composition of claim 8, wherein said high-intensity sweetener is stevia, said fiber is nutriose and said alcohol-based sweetener is erythritol.

10. The composition of claim 1, is in a form of soft candy.

11. The composition of claim 10, wherein said form of soft candy is in a form selected from a group comprising, a chewy candy, a gummy candy, or a jelly candy.

12. The composition of claim 1, is in a form of hard candy.

13. The composition of claim 12, wherein said form of hard candy is in a form of lollypop type candy.

14. The composition of claim 1, is in a form of a powder.

15. The composition of claim 14, wherein said form of powder can be added to water to achieve same therapeutic results.

16. A non-acidic-based composition for improving oral health and delivering nutrients, therapeutic agents, or combinations thereof, comprising:
at least one fluoride-based compound;
at least one calcium-based compound;
at least one acid neutralizing agent;
at least one active ingredient;
at least one tooth whitening agent;
at least one probiotic;
at least one energy supplement;
at least one sweetener;
at least one hydrating agent;
at least one additive, and
at least one agent to assist with halitosis,
wherein said composition is configured to neutralize pH in an oral cavity and delivers ions to create an environment that arrests or reverses disease processes.

17. The composition of claim 16, wherein said additive is selected from a group comprising an antimicrobial agent, an antibacterial agent, an antiviral agent, a colorant, and a flavorant.

18. The composition of claim 16, further comprises at least one vitamin or mineral.

19. The composition of claim 16, further comprises at least one electrolyte.

20. The composition of claim 16, wherein said at least one agent to assist with halitosis, is lactobacillus.

21. The composition of claim 16, wherein said acid neutralizing agent is calcium bicarbonate.

22. The composition of claim 16, further comprises a stimulant, wherein said stimulant is caffeine.

23. The composition of claim 16, provides the best environment for remineralization, wherein the candy composition comprises of a high-intensity sweetener in a range of about 0.001 g to about 0.006 g, fiber in the range of about 2-6 g, β-tricalcium phosphate (β-TCP) in a range of about 0.5 g-5 g, alcohol-based sweetener in the range of about 1-6 g, fluoride-based compound ranging from 0 g to about 0.2 g, bicarbonate in the range of about 0.02 mg to about 20 mg and water.

24. The composition of claim 23, wherein said high-intensity sweetener is stevia, said fiber is nutriose and said alcohol-based sweetener is erythritol.

25. The composition of claim 16, is in a form of soft candy.

26. The composition of claim 25, wherein said form of soft candy is in a form selected from a group comprising, a chewy candy, a gummy candy, or a jelly candy.

27. The composition of claim 16, is in a form of hard candy.

28. The composition of claim 27, wherein said form of hard candy is in a form of lollypop type candy.

29. The composition of claim 16, is in a form of a powder.

30. The composition of claim 29, wherein said form of powder can be added to water to achieve same therapeutic results.

31. A non-acidic-based composition for improving oral health and delivering nutrients, therapeutic agents, or combinations thereof, wherein said composition is configured to neutralize pH in the oral cavity and delivers therapeutic agents to create an oral environment that reverses disease processes in the oral cavity and improves general health.
